(19) United States
(12) Patent Application Publication
(10) Pub. No.: US 2021/0198791 A1
(43) Pub. Date: Jul. 1, 2021

(54) Portable Hydrogen Ventilator

(71) Applicant: NG TAI CHEUNG, YUEN LONG (HK)

(72) Inventor: NG TAI CHEUNG, YUEN LONG (HK)

(21) Appl. No.: 16/731,371

(22) Filed: Dec. 31, 2019

(51) Int. Cl. C25B 1/04; C25B 15/08; C25B 15/02; A61M 16/10; A61M 16/00

(52) U.S. Cl. CPC C25B 1/04; C25B 15/08; H01M 16/00; A61M 16/107; A61M 16/022; C25B 15/02

(57) ABSTRACT

Embodiments relates to an advance type of portable hydrogen ventilator, which mainly includes: main body, main battery, function board, electrolytic sheet, electrolytic cell and end cover; wherein the bottom of the main body is provided with a battery, the side is provided with a function key, and the top of the main body is closed by an end cover; one side of the function board is connected with a battery, the other side is connected with an electrolytic sheet, and the function board is provided with a function Key; the electrolytic piece is fixed on the main body and placed in an electrolytic cell; the top of the electrolytic cell is provided with a hydrogen filtering chamber. By fixing the electrolytic sheet on the main body, the utility model is convenient for disassembly and replacement, and the electrolysis process is better controlled through the cooperation of the function key and the function sheet. At the same time, the problem of electrolyte leakage is avoided; the utility model has the advantages of simple structure, high gas production quality, convenient operation and low cost.

### BACKGROUND

### Field of the Invention

The present invention relates to medical and health-care products technical field, especially a kind of portable hydrogen ventilator.

### Description of the Related Art

Hydrogen molecule has the function of anti-oxidation and neutralizing free radicals, which is suitable for clinical treatment of causes, such as insomnia, constipation, chronic diseases (such as hypertension, hyperlipidemia, hyperglycemia, etc.), and even rehabilitation treatment of cancer. Therefore, in order to carry out treatment, patients using hydrogen hydrogen breathing machine arises at the historic moment, However, the existing hydrogen equipment is either very large, because it cannot be carried around, and it is not suitable for patients to use it at all, and the hydrogen produced by the existing hydrogen ventilator is a continuous supply. Causes the patient to inhale half and waste half: the patient can only use hydrogen effectively when inhaling, but when the patient is exhaling, hydrogen can only be wasted. And at present, the hydrogen engine on the market uses the form of electrolyte, and when it is used, the leakage of the electrolyte is prone to occur, resulting in safety

Previous hydrogen gas supply system, which stores hydrogen gas type, gas and liquid, mostly two forms. Because of gaseous hydrogen, liquid hydrogen has flammable properties, therefore, the previous hydrogen supply system will be worrying danger of explosion. To reduce the risk of previous hydrogen supply system, the previous hydrogen supply system would be firmly fixed to prevent dumping of causing an explosion, and therefore, the previous hydrogen supply system cannot be designed as a portable hydrogen supply system.

There are multiple inventions that have been designed in prior art regarding portable hydrogen ventilator. For instance, a kind of portable hydrogen ventilator bearing Chinese patent 1,098,52986A is issued to Chinses inventor. The invention discloses a kind of portable hydrogen ventilators, comprising: body, body are equipped with electrolysis chamber, water supply chamber, hydrogen gas storage chamber and gas control valve; Water supply chamber and electrolysis chamber, and body is equipped with the inlet pipeline for supplying water to electrolysis chamber; Flow through hydrogen gas storage chamber in the intermittent supply of gas control valve by the hydrogen that electrolysis chamber generates: when gas control valve is in close state, hydrogen is stored in hydrogen gas storage chamber; When gas control valve is in the open state, hydrogen is flowed out through gas control valve. Hydrogen ventilator of the present invention both can be applied to house health, it is more convenient travel outdoors in carry use, substantially increase the scope of application and ease of use of hydrogen ventilator. More preferably, hydrogen utilization ratio is being improved, while avoiding hydrogen from wasting, is also improving the unit time supply amount of this hydrogen ventilator.

Another patent on a kind of portable electrolysis hydrogen lung ventilator bearing U.S. Pat. No. 1,087,14261A is issued to Chinese inventor. The patent discusses a kind of portable electrolysis hydrogen lung ventilator, belongs to water electrolysis hydrogen production technical field, and ontology is kettle type, is made of engine base, electrode module, control system, battery, upper shell and pedestal; Electrode module is hemispherical, mounted on the inside cavity of ontology, under the action of proton exchange membrane, by body cavity internal body portion at cathode space and anode compartment, to which hydrogen and oxygen caused by electrolysis are separated, the oxygen tapping equipment that oxygen arrives bodies top by the gas nozzle and tracheae that are electrolysed die set top again discharges; Hydrogen is then directly exported by the hydrogen output device connecting gas transmission pipe of bodies top from cathode space and is sucked to human body nasal cavity, and the health care of human body and the rehabilitation of disease are conducive to. Have the characteristics that small and exquisite, easy to carry, simple and practical, the clean health of type, safe and reliable, operation is efficient.

A Korean patent 2,016,0137208A on Portable Hydrogen and Hydrowater Ganerater is issued to Korean inventor. The present invention relates to an apparatus for producing a portable functional water having a hydrogen respiratory function and in which power supplied from the power supply unit 111 is supplied to the cathode electrode unit 113 located on the upper side and the anode electrode unit 113 located on the lower side through the main control unit 112, A cation exchange membrane 115 is stacked and inserted between the cathode electrode unit 113 and the anode electrode unit 114 so that water is supplied to the cathode electrode unit 113 A hydrogen generator 110 capable of generating hydrogen gas from the cathode electrode unit 113 and a cathode electrode unit 113 connected detachably to the upper side of the hydrogen generator 110, The hydrogen gas is passed through the cannula (141), and the hydrogen gas is passed through the water pipe (120). The water pipe Supplying cannulas 140; And a portable respiratory water supply device (100) having a hydrogen respiratory function.

Another Water hydrogen breathing machine bearing Chinese patent 1,051,94779A is issued to Chinese inventor. The invention discloses a water hydrogen breathing machine. The water hydrogen breathing machine comprises a breathing machine body, a methanol-utilizing hydrogen production system and a hydrogen-utilizing electricity generation system. The methanol-utilizing hydrogen production system, the hydrogen-utilizing electricity generation system and the breathing machine body are connected in sequence. The breathing machine comprises an air source, an air-oxygen mixer, a mainframe, a humidifying device and an external pipeline. The methanol-utilizing hydrogen production system produces hydrogen through methanol steam reforming, high-purity hydrogen is obtained through a membrane separation device plated with palladium-silver alloy, the hydrogenutilizing electricity generation system utilizes the obtained hydrogen to generate electricity, and generated electric energy is supplied to the breathing machine body for work. The water hydrogen breathing machine can utilize methanol to produce hydrogen so as to generate electricity serving as energy of the breathing machine and can be used in a place without alternating current so that the breathing machine can be used in the field of mobile medical treatment, and the medical care quality of mobile medical treatment can be improved.

A Portable hydrogen supplying system bearing Chinese patent 1,755,186A is issued to Chinese inventor. The invention provides a portable hydrogen-donating system to supply hydrogen by gas pattern for a hydrogen appliance device, which comprises the following parts: at least one control valve, inlet and outlet. When hydrogen-donating system sucks hydrogen into each hydrogen storing pot and connects the storing pot, the system supplies stable pressure hydrogen to hydrogen appliance, which can be adjusted by the control valve.

There are multiple solutions that have been presented in prior art. However, these solutions are limited and restricted to their conventional systems. Moreover, the current solutions that exist in the marketplace today, aren't cost effective and aren't convenient for every user. Therefore, a need to provide a hydrogen ventilator with convenient use, high safety performance and low cost is an urgent problem for technicians in this field.

The current invention proposes a portable hydrogen ventilator which is convenient to use, with high safety performance and low cost.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of portable hydrogen ventilator.

The main objective of the invention is to provide an improved assembly which is highly portable and is convenient to use, with high safety performance.

It is further the objective of the invention to provide a portable hydrogen ventilator which is lower in cost then other similar available ventilators.

It is also the objective of the invention to provide a hydrogen ventilator both can be applied to occupy family's health and is more convenient travel outdoors in carry use, substantially increase the scope of application of hydrogen ventilator and easy-to-use Property

It is a further object of the invention to enhance the hydrogen utilization ratio, while avoiding hydrogen from wasting, the unit time for this hydrogen ventilator that also double the supply amount.

It is further the objective of the invention to provide a quick adjustable assembly which can be easily utilized at any place and with the control key assembly with a start key, an open key and a USB charging interface can be utilized easily.

Thus, it is the objective to provide a new and improved solution for portable hydrogen ventilator. It has an added advantage of simple structure, high gas production quality, convenient operation and low cost. Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWINGS

FIG. 1 is a schematic diagram of the structural explosion of the utility model.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to provide an effective methodology to provide portable hydrogen ventilator which is convenient to use, with high safety performance and low cost.

In view of this, the utility model provides a new portable hydrogen ventilator which is convenient to use, has high safety performance and low cost.

To achieve the above purposes, the utility model provides as per its preferred embodiments the following technical scheme, which mainly includes: main body, main battery, function board, electrolytic sheet, electrolytic cell and end cover; The bottom of the main body is provided with a battery, the side is provided with a function key, and the top of the main body is closed by an end cover; One side of the function board is connected with a battery, the other side is connected with an electrolytic sheet, and the function board is provided with a function key; The electrolytic piece is fixed on the main body and placed in an electrolytic tank; The top of the electrolytic cell is provided with a hydrogen filtering tank.

As per additional embodiments, the new portable hydrogen ventilator, the end cover is provided with a nozzle and a feeding port, and the end cover is provided with a perforating hole inside, so that the nozzle is connected to the hydrogen filtering tank; The feeding port extends into the electrolysis tank.

Preferably, in a new type of portable hydrogen ventilator, the bottom of the hydrogen filter chamber is provided with a separator and the inside is provided with a filtering cotton.

The hydrogen filtering tank as per its further embodiments can not only make the hydrogen produced high quality, but also avoid the problem of electrolyte leakage. The utility model is characterized by simple structure, high quality of gas production, convenient operation and low cost.

In order to explain the embodiment of the utility model or the technical solution in the prior art more clearly, the following will give a brief introduction to the drawings needed in the embodiment or the prior art description. It is obvious that the drawings in the following description are only the embodiments of the utility model. For the general technical personnel in this field, under the premise of not paying creative labor, other appended drawings can be obtained according to the provided appended drawings.

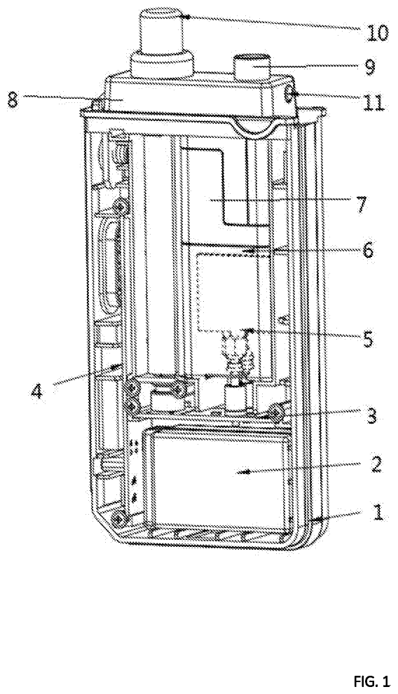

FIG. 1 is a structural diagram of the utility model.

A clear and complete description of the technical scheme of the embodiments of the utility model will be given below by combining the appended drawings of the embodiments of the utility model. Obviously, the embodiments described are only a part of the embodiments of the utility model, but not all of the embodiments. Based on the embodiments in the utility model, all other embodiments obtained by ordinary technicians in the field without creative labor are within the scope of the protection of the utility model.

As referred to attached FIG. 1, which discloses a new portable hydrogen ventilator for the utility model.

The utility model mainly includes: main body 1. Battery 2. Function board 3. Electrolytic slice 5. Electrolytic bin 6. End cover 8; wherein the bottom of the main body 1 is provided with battery 2. The side is provided with function key 4. And the top of the main body 1 is closed by end cover 8; one side of the function board is connected to battery 2. And the other side is connected to electrolytic sheet 5. Meanwhile, the function board 3 is provided with function key 4; the electrolytic sheet 5 is fixed on the main body 1 and placed in the electrolytic bin 6; The top of the electrolytic tank 6 is provided with a hydrogen filtering tank 7.

In order to further optimize the above technical scheme, nozzle 10 and feeding port 9 are arranged on the end cover, and perforating hole 11 is arranged inside the end cover 8, so that nozzle 10 is connected to hydrogen filtering tank 7. The feeding port 9 extends into the electrolytic tank 6.

In order to further optimize the above technical scheme, hydrogen filtering tank 7 is equipped with a partition at the bottom and filtering cotton inside.

In order to further optimize the above technical scheme, battery power supply for function board 3, 2 4 through function keys to control the process of electrolysis, may at any time and interrupt the electrolytic process, at the same time to control current and control degree of electrolysis, electrolytic piece fixed on the main body 1, 5 when electrolytic piece five consumption is too large, can change at any time; The hydrogen produced by electrolysis flows from the electrolysis cell 7 to the nozzle 10 in the end cover 8, in which the hydrogen is filtered through the hydrogen filtering tank 7 to filter the water and impurities in the hydrogen, making the quality of hydrogen better.

Each embodiment in this specification is described in a progressive manner. Each embodiment highlights the differences with other embodiments, and the same and similar parts between each embodiment can be referred to each other. For the device disclosed by an embodiment, the description is relatively simple as it corresponds to the method disclosed by the embodiment. For the relevant points, please refer to the description in the method section.

The above description of the disclosed embodiments enables professionals in the field to realize or use the utility model. Multiple modifications to these embodiments will be apparent to professionals in the field, and the general principles defined in this article may be implemented in other embodiments without deviating from the spirit or scope of the utility model. Therefore, the utility model will not be limited to the embodiments shown herein, but will conform to the widest range consistent with the principles and novel features disclosed herein.

Each embodiment in this specification is described in a progressive manner. Each embodiment highlights the differences with other embodiments, and the same and similar parts between each embodiment can be referred to each other. For the device disclosed by an embodiment, the description is relatively simple as it corresponds to the method disclosed by the embodiment. For the relevant points, please refer to the description in the method section.

The above description of the disclosed embodiments enables professionals in the field to realize or use the utility model. Multiple modifications to these embodiments will be apparent to professionals in the field, and the general principles defined in this article may be implemented in other embodiments without deviating from the spirit or scope of the utility model. Therefore, the utility model will not be limited to the embodiments shown herein but will conform to the widest range consistent with the principles and novel features disclosed herein.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. The utility model relates to a new type of portable hydrogen ventilator, which is characterized in that it includes:
main body; main battery; function board; electrolytic sheet; electrolytic cell; end cover; bottom of main body provided with a battery; side provided with a function key and top of the main body is closed by an end cover. [0045] claim II: A new type of portable hydrogen ventilator where one side of the function board is connected with a battery, the other side is connected with an electrolytic sheet, and the function board is provided with a function key; The electrolytic sheet is fixed on the main body and placed in an electrolytic cell; A hydrogen filtering tank is provided on the top of the electrolytic tank.
According to the new portable hydrogen ventilator mentioned in claim 1, its characteristics are that the end cover is provided with a nozzle and a feeding port, and the inside of the end cover is provided with a perforating hole to connect the nozzle to the hydrogen filtering tank; The feeding port extends into the electrolysis tank.

2. According to the new portable hydrogen ventilator mentioned in claim 1, its characteristics lie in that the bottom of the hydrogen filtering bank is provided with a partition and the inside is provided with a filtering cotton.
